(19) United States
(12) Patent Application Publication
(10) Pub. No.: US 2018/0342178 A1
(43) Pub. Date: Nov. 29, 2018

(54) CARDIOPULMONARY RESUSCITATION TRAINING APPARATUS AND METHOD

(71) Applicant: Steven A. GOLD, Tiburon, CA (US)

(72) Inventor: Steven A. GOLD, Tiburon, CA (US)

(21) Appl. No.: 15/987,873

(22) Filed: May 23, 2018

Related U.S. Application Data
(63) Continuation-in-part of application No. 15/602,594, filed on May 23, 2017.

(51) Int. Cl. G09B 23/28; A61B 5/107; G09B 5/02; G09B 5/04

(52) U.S. Cl. CPC G09B 23/288; G09B 5/04; G09B 5/02; A61B 5/1075

(57) ABSTRACT

A method for cardiopulmonary resuscitation training includes sensing a chest compression force via a distributed sensor, measuring a depth of the chest compression via an accelerometer, calculating depth of the chest compression based at least in part on the sensed chest compression force and, in some cases, on the measured depth of the chest compression.

### FIELD OF THE INVENTION

The present disclosure relates generally to training devices and particularly to a cardiopulmonary resuscitation (CPR) training apparatus. More particularly, the present disclosure relates to a CPR training apparatus that senses chest compression force throughout a chest compression area and provides performance information including an actual location of the exerted chest compression force over the chest compression area.

### BACKGROUND

Cardiopulmonary resuscitation (CPR) is a lifesaving artistry used in exigent circumstances involving cardiac arrest due to heart attacks, asphyxiation, etc. During cardiac arrest, blood no longer receives the necessary oxygen. Lack of oxygenated blood, in turn, may result in brain damage or death to the person (i.e., the patient) within only a few minutes unless remedial measures are taken. CPR may be applied to induce blood flow from and to the heart and circulation of oxygenized blood.

The American Heart Association suggests that CPR should begin immediately upon cardiac arrest. The person administering CPR should first apply 30 chest compressions. Each chest compression involves pressing down on the chest approximately 5 cm and totally releasing before commencing a subsequent chest compression. Such compression and release may induce blood flow to and from the heart until spontaneous circulation returns (i.e., the patient's heart starts to beat autonomously). Then, the patient's airway should be checked. If normal breathing has not returned, rescue breathing (e.g., two mouth-to-mouth breaths after 30 chest compressions) must be administered. Then, the process repeats. Administering CPR may be the only remedial measure available until additional medical treatment arrives at the scene. Hence, proper and effective administration of CPR may be essential in saving a person's life. People must be trained to administer CPR properly.

The location or angle at which the CPR compression force is applied to the patient is important. Compression force must be applied directly (i.e., 90 degree angle) over the patient's chest. Force applied at improper locations or angles may be ineffective or worse. Conventional CPR training devices cannot provide feedback to the trainee regarding location or angle of the applied chest compression force.

Moreover, conventional CPR training devices cannot provide accurate feedback regarding depth of chest compression, particularly when the patient is lying on a soft or compliant surface such as a bed. This is a significant limitation because, often, the patient may be on a hospital bed.

### SUMMARY OF THE INVENTION

The CPR training apparatus and methods disclosed herein allow sensing of chest compression force applied throughout the chest compression area and specifically allow for sensing of the location or angle at which the force is applied. This way a CPR trainee may accurately and effectively recognize correct chest compression parameters including proper location or angle. The apparatus provides the CPR trainee important performance information that conventional CPR devices do not provide. While other devices register depth of compressions, release and rate, no other device registers angle of compressions. Due to sensors distributed throughout the pad disclosed herein, the device is able to determine angle of compressions. Compressions at an angle (i.e., other than a right angle directly above the chest) do not effectively push down on the heart and are wasted effort.

Conventional CPR devices tend to be limited in the information they provide to the CPR administrators. Those devices tend to use sensors (e.g., an accelerometer, force sensor, pressure sensor, velocity sensor, etc.), which individually or collectively detect an amount, velocity, or frequency of force applied for chest compressions. Such information can be useful in determining an instantaneous depth of the chest compressions. However, a proper and effective CPR is not determined based only on the depth of the chest compressions, especially if the compression is not based on vertical pressure. For example, as described above, correct location or angle of the force applied is an important factor in administering proper and effective CPR.

Understanding the actual versus the correct location of the chest compression force during CPR is crucial. The force must be applied over a specific chest area (i.e., the sternum of the patient) at which the resulting compression may induce oxygenized blood circulation. By providing feedback regarding correct versus actual location of the applied chest compression force, the apparatus and method disclosed herein allow the CPR trainee to correctly realign the chest compression force to be over the sternum for administering proper and effective CPR. Therefore, the present disclosure provides a more effective CPR training apparatus and method as compared to conventional CPR devices, which fail to provide actual versus correct location information of the chest compression force.

Moreover, relying solely on an accelerometer for correct compression measurement may be deficient. For example, when the patient is on a soft surface such as a bed, an accelerometer may give a false reading, as the bed is compressing, not the chest. The present disclosure registers force and depth of compression. If the patient is on a soft surface and distance traveled during the compression exceeds a predetermined amount, the sensor ignores the accelerometer data and utilizes force applied to the patient's chest as the determining factor for proper CPR compression.

These and further features of the present invention will be described with reference to the attached drawings. In the description and drawings, particular embodiments of the invention have been disclosed in detail as being indicative of some of the ways in which the principles of the invention may be employed, but it is understood that the invention is not limited correspondingly in scope. Rather, the invention includes all changes, modifications and equivalents coming within the terms of the appended claims.

### BRIEF DESCRIPTION OF THE DRAWINGS

The accompanying drawings, which are incorporated in and constitute a part of the specification, illustrate various example systems, methods, and so on, that illustrate various example embodiments of aspects of the invention. It will be appreciated that the illustrated element boundaries (e.g., boxes, groups of boxes, or other shapes) in the figures represent one example of the boundaries. One of ordinary skill in the art will appreciate that one element may be designed as multiple elements or that multiple elements may be designed as one element. An element shown as an internal component of another element may be implemented as an external component and vice versa. Furthermore, elements may not be drawn to scale.

FIG. 1 illustrates a block diagram of an exemplary CPR training apparatus.

FIGS. 2A and 2B illustrate top and bottom views, respectively, of an exemplary pad for the CPR training apparatus.

FIG. 2C illustrates a transparent view of a patient's chest.

FIGS. 3A-3C illustrate front, side, and rear views of an exemplary distributed sensor of the exemplary CPR training apparatus.

FIG. 3D illustrates an inner view of the exemplary distributed sensor of the exemplary CPR apparatus.

FIG. 3E illustrates an inner view of a second exemplary distributed sensor of the exemplary CPR training apparatus.

FIG. 3F illustrates a front view of a third exemplary distributed sensor of the exemplary CPR training apparatus.

FIG. 4A illustrates a proper location of chest compression force applied throughout a surface area of a pad in which an exemplary distributed sensor is uniformly distributed.

FIG. 4AA illustrates CPR technique corresponding to the location of force sensed in FIG. 4A.

FIGS. 4B and 4C illustrate respective improper locations of the chest compression force throughout the surface area of the pad.

FIGS. 4BB and 4CC illustrate CPR techniques corresponding to the locations of force sensed in FIGS. 4B and 4C, respectively.

FIGS. 4D and 4E illustrate respective improper angles or hand form of the chest compression force throughout the surface area of the pad.

FIGS. 4DD and 4EE illustrate CPR techniques corresponding to the locations of force sensed in FIGS. 4D and 4E, respectively.

FIG. 5 illustrates the exemplary CPR training apparatus of FIG. 1 in use on a patient.

### DETAILED DESCRIPTION

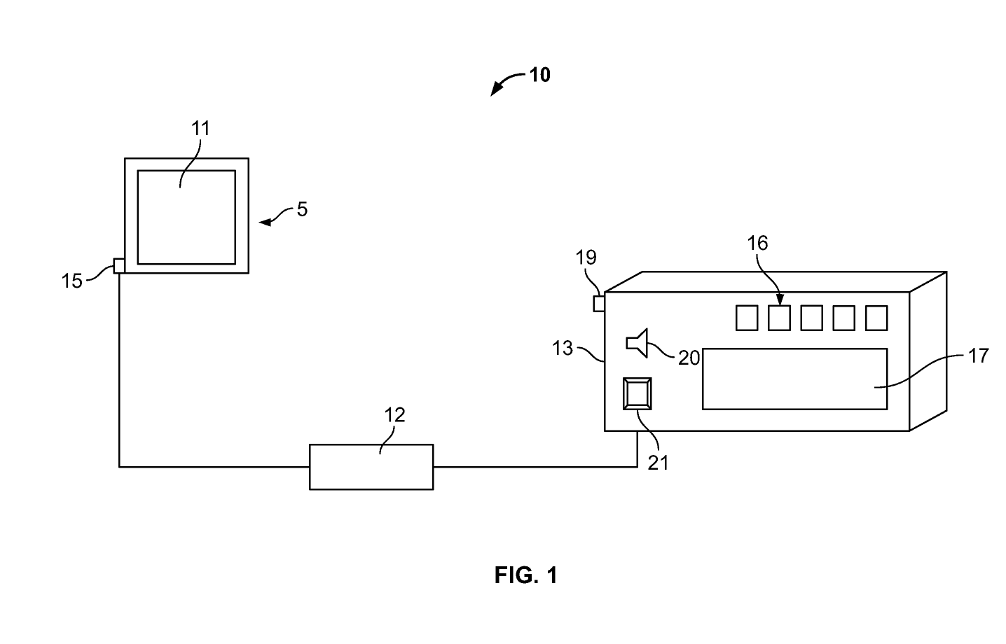

FIG. 1 illustrates a block diagram of an exemplary CPR training apparatus 10. The CPR training apparatus 10 may include a pad 5 that has a distributed sensor 11. The CPR training apparatus 10 may also include a controller 12 and a prompting device 13. Although for illustration purposes the controller 12 is shown independent of the prompting device 13, in some embodiments, the controller 12 may reside with the prompting device 13. The distributed sensor 11 senses chest compression force applied during chest compressions. The controller 12 is operatively coupled to the distributed sensor 11 and the prompting device 13 and controls the distributed sensor 11 and prompting device 13 when the CPR training apparatus is in use. The pad 5 may, for example, include a connector 15 to connect the pad 5 to the controller 12 or prompting device 13. The prompting device 13 provides a CPR trainee information necessary for administering proper and accurate CPR.

The distributed sensor 11 is uniformly distributed on the pad 5 which has at least 91 cm² surface area to accommodate a person's hand and is configured to sense chest compression force applied throughout the surface area. In one embodiment, the pad 5 may have dimensions of 9.5 cm length×9.5 cm width×0.125 cm depth. The distributed sensor 11 may be distributed over the front surface area or inner surface area of the pad 5. The pad 5 may, for example, correspond to fabric produced by BeBop Sensors of Berkeley, Calif. The distributed sensor 11 senses the chest compression force throughout the surface area of the pad 5 when a trainee administers a chest compression, including sensing if more pressure is applied to one area vs another. Thus, the distributed sensor 11 is able to register if pressure is being applied at an angle rather than directly over the patient. The distributed sensors may operate in accordance with the operational detail disclosed in a U.S. Pat. No. 9,076,419 hereby incorporated by reference in its entirety. Upon sensing the chest compression force applied, the distributed sensor 11 transmits a signal including the sensed compression force to the controller 12.

The controller 12 (e.g., a microprocessor, a central processing unit of a computing device, or an integrated chip) is operatively connected to the distributed sensor 11 and the prompting device 13. The controller 12 receives the sensed chest compression force from the distributed sensor 11. The controller 12, in turn, calculates a chest compression parameter based at least in part on the sensed chest compression force throughout the surface area. A chest compression parameter may include a velocity, frequency, amount, or location of the sensed chest compression force. A chest compression parameter may also include vertical distance or depth of the chest compression. The controller 12 may then compare the calculated chest compression parameter with a predefined value for the chest compression parameter to output performance information such as, for example, too much force, not enough force, incorrect location, insufficient depth, insufficient release, etc.

Predefined values may correspond to values that the American Heart Association (AHA) recommends. For example, for an adult patient suffering from cardiac arrest, CPR force should be applied at the center of the chest, above the sternum (“where the ribs come together”). The target compression location may be stored to be compared to actual chest compression location during CPR training. During training, based on the comparison result, the controller 12 produces performance information, which may indicate whether the sensed chest compression location is adequate or not.

As described above, understanding the location of the chest compression force throughout the surface area is essential in administering proper and effective CPR. The training apparatus 10 may determine location of the sensed chest compression force throughout the surface area by locating a center of mass of the trainee's exerted force over the surface area of the pad 5. A center of mass is a defined term and may mean an arithmetic mean of all points weighted by a local density or specific weight. A correct location for the chest compression force to be applied is a point (also referred to as a target sternum point) just above of the patient's sternum, at which the lower rib cages meet. Hence, the center of the mass of the trainee's exerted force and the target sternum point should be vertically aligned for proper, accurate and effective CPR. Any significant misalignment between the center of mass of the trainee's exerted force and the target sternum point may simply waste the force applied and deprive the patient of the life-saving force or, worse yet, may further injure the patient.

In order to facilitate a correct alignment of the center of the mass and the target sternum point, the pad 5 should be placed over the patient's chest area so that the center of the surface area of the pad is also vertically aligned with the target sternum point. Thus, when the sensed chest compression force is displayed by the prompting device 13, the trainee may achieve the correct vertical alignment by simply moving the actual location of the chest compression force towards the center of the surface area.

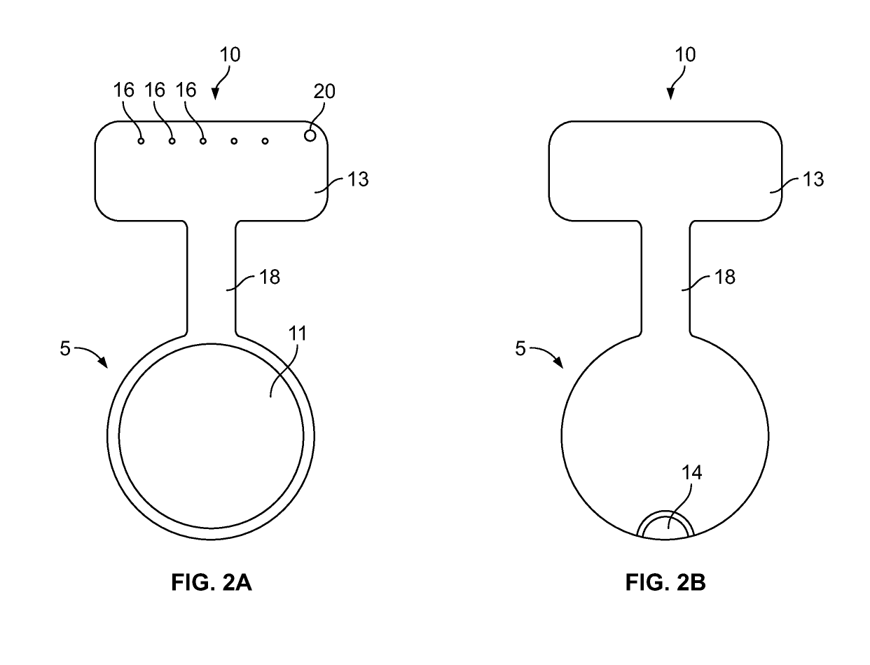

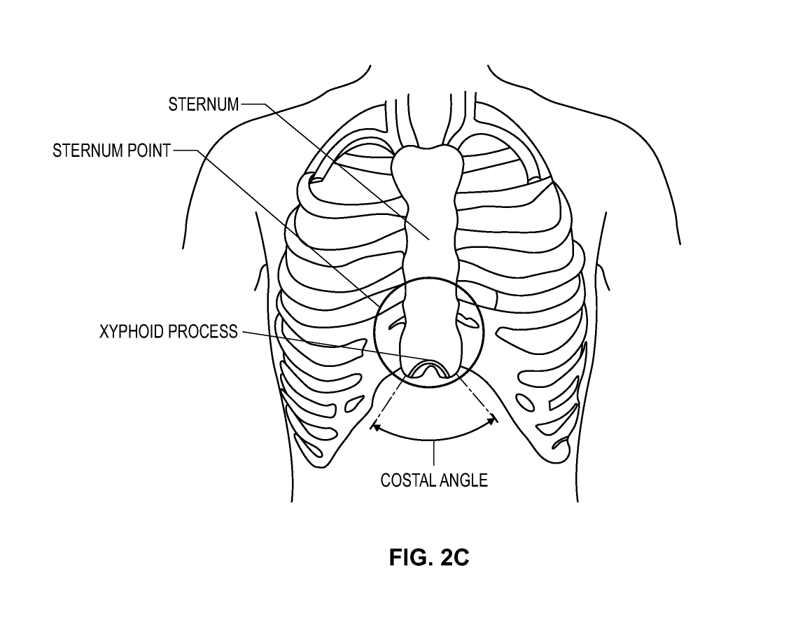

FIGS. 2A and 2B illustrate top and bottom views, respectively, of an exemplary pad 5. The pad 5 includes the distributed sensor 11 and may include a placement aide 14 formed on the bottom side of the pad 5 and shaped similar to a positive image of a person's costal angle of the xyphoid process of the sternum or chest. FIG. 2C illustrates a transparent view of a patient's chest and particularly the sternum ST whose shape the placement aide 14 mimics. Specifically, the placement aide 14 fits below the sternum ST and has a shape generally corresponding to that of the costal angle CA of the xyphoid process XP of the sternum ST. The placement aide 14 may be used to engage the costal angle CA of the xyphoid process XP of the sternum ST thus engaging pad 5 in place and facilitating consistently placing the pad 5 over the patient's chest area so that the center of the surface area of the pad 5 is vertically aligned with the target sternum point SP.

In the embodiment of FIGS. 2A and 2B, the pad 5 and the prompting device 13 are part of single structure. The pad 5 and the prompting device 13 are joined by a bridge 18 which may also incorporate electrical connections between the pad 5 and the prompting device 13. The prompting device 13 may include light emitting elements 16 and audio device 20 for notifying the trainee of the performance information during chest compressions. Although not shown in FIGS. 2A and 2B, the prompting device 13 may also incorporate the controller 12.

The controller 12 produces the performance information, which may further include any corrective measure to be taken by the trainee. For example, if a total recoil of the chest has not occurred, the prompting device 13 may alert via the display 17 to completely release pressure or the audio device 20 may alert the trainee to wait before applying another chest compression or increase rate of compression. The performance information may further include an instruction (e.g., when to administer rescue breathing) based on the calculated frequency of the chest compressions. The prompting device 13 is operatively coupled to the controller 12 and is configured to communicate to the trainee the performance information produced by the controller 12. The prompting device 13 may include light emitting elements 16 for notifying the trainee of the performance information during chest compressions.

The light emitting elements 16 may include various LED of different colors. Each light emitting element 16 indicates respective performance information based on the comparison. For example, a light emitting element 16 may indicate performance information regarding the amount of the sensed chest compression force throughout the surface area. A proper amount (also referred to as a predefined amount) of chest compression force for an adult is approximately 29.5 kg. If the calculated amount of the sensed compression force is lower, for example 27 kg, corresponding performance information includes that the amount of the sensed chest compression force throughout the surface area is less than the predefined amount.

Based on the performance information, a yellow light emitting element 16 may be lit indicating that the amount of the sensed chest compression force throughout the surface area is less than the predefined amount of chest compression force to be applied for a chest compression. If the calculated amount of the sensed chest compression force is larger than 29.5 kg, a red light emitting element 16 may be lit indicating the amount of the sensed chest compression force throughout the surface area is more than the predefined amount of the chest compression force to be applied for the chest compression. If the calculated amount of the sensed chest compression force is equal to 29.5 kg, a green light emitting element 16 may be lit indicating the amount of the sensed chest compression force throughout the surface area is equal to the predefined amount of the chest compression force to be applied for the chest compression. If the calculated amount of the sensed chest compression force is zero, a blue light emitting element 16 may be lit to indicate the amount of the sensed chest compression force throughout the surface area is zero. A zero amount of the force applied means a total recoil after a chest compression. A total recoil is necessary for refilling of the heart with oxidized blood between the chest compressions.

The light emitting element 16 may also include a white light emitting element 16, which may turn on to indicate a point in time for administering rescue breathing during chest compressions. The number of the light emitting elements 16 is not limited to five and can vary depending on a preference or need of a user. Further, the light emitting element(s) 16 may also be arranged on the surface area of the pad 5 (see FIGS. 2A and 2B). The light emitting element(s) 16 arranged on the surface area of the pad 5 may perform the same or different functions as the light emitting element 16 arranged on the prompting device 13.

The prompting device 13 may also include a display 17 for presenting performance information produced by the controller 12. For example, the display 17 may display the actual location of the sensed chest compression force with respect to the center of the surface area of the pad. The center of the surface area of the pad 5 should be vertically aligned with the target sternum point at which the lower rib cages of the patient meet. The placement aid 14 on the underside of pad 5 assists in proper placement of pad 5. At that position, the center of the surface area of the pad 5 corresponds to the recommended location for exerting compression force. Any force exerted significantly outside the center of the surface area of the pad 5 is being applied at an incorrect location or angle. The sensors in pad 5 register if pressure is applied at an angle and not vertically. With this information, the trainee can make an appropriate corrective measure by simply moving his hands towards the center of the surface area of the pad 5 or changing the angle of compressions. The display 17 may include a touchpad, which can be interfaced by the trainee to perform other relevant operations (e.g., storing a performance information for use in reviewing performance improvement of the trainee) using the CPR training device 13.

The prompting device 13 may also include an audio device 20 (e.g., a speaker). The audio device 20 may indicate actual frequency of chest compressions as compared to a predefined recommended frequency of chest compressions. For instance, the American Heart Association recommends 120 chest compressions per minute (also referred to as a recommended frequency) to be applied for an adult patient. If the controller 12 calculates a frequency of chest compressions substantially equal to the predefined recommended frequency, then the controller produces performance information to that effect. The audio device 20 may release, for example, a first pitch. If the controller 12 calculates a frequency of chest compressions significantly higher than the predefined recommended frequency, then the controller 12 may produce respective performance information to that effect. The audio device 20 may release, for example, a second pitch higher in pitch than the first pitch. If the controller 12 calculates a frequency of chest compressions lower than the predefined frequency, the controller 12 produces corresponding performance information to that effect. The audio device 20 may then release a third pitch lower than the first pitch. The audio device 12 is not limited to indicating the rate of chest compressions. For instance, the audio device 12 may announce corrective information (e.g., “move the hands to the right by 2 cm”) for the trainee to hear during chest compressions.

In reference to FIG. 1, the device 10 may include a USB or similar port 19 that may connect to a computer to download data regarding a CPR incident or training session such as the depth of compressions, if total release occurred, rate of compressions, location of compressions, angle of compressions, etc. The device 10 or an external computer may keep a log of this data for later evaluation. Researchers and practitioners may use this data, including location and angle of compression, to determine if proper CPR has been performed. The prompting device 13 may also include a switch 21 for turning on and off the prompting device 21.

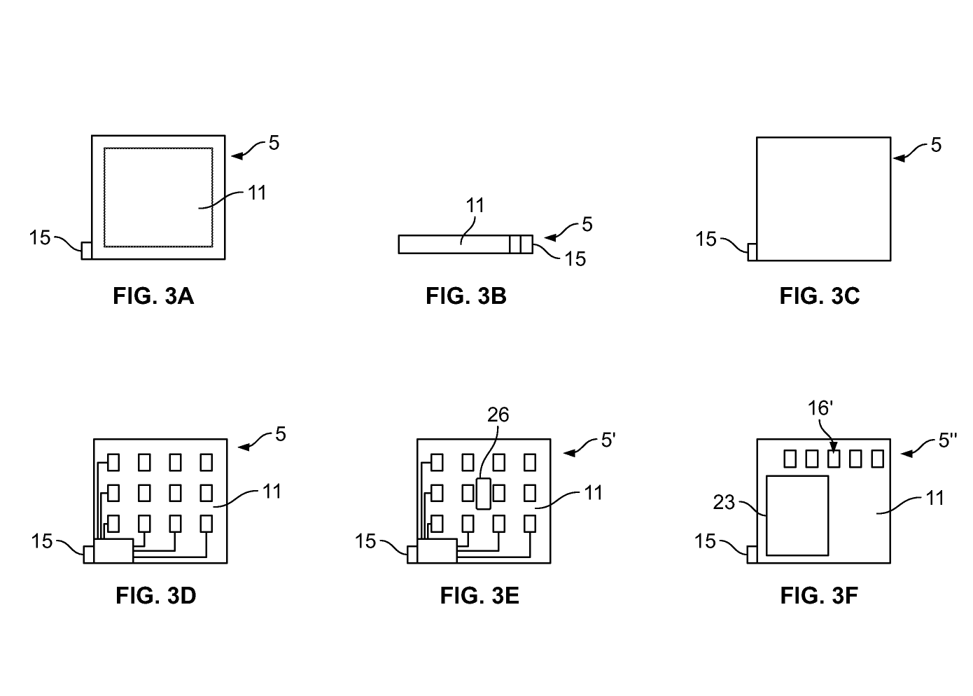

FIGS. 3A-3C illustrate front, side, and rear views of an exemplary pad 5 of the exemplary CPR training apparatus 10. The pad 5 includes a distributed sensor 11 that is uniformly distributed over the pad 5, which has at least 91 cm² surface area. The pad 5 should have dimension that accommodate a person's hand. The pad 5 may include a fabric produced by BeBop Sensors of Berkeley, Calif. The distributed sensor 11 senses the chest compression force throughout the surface area when a trainee applies a chest compression. The distributed sensor 11 may operate in accordance with the operational detail disclosed in a U.S. Pat. No. 9,076,419 (hereby incorporated by reference in its entirety). Upon sensing the chest compression force applied, the distributed sensor 11 transmits a signal including the sensed compression force to the controller 12. The pad 5 may also include a connector 15 that may be connected to the controller 12 via a cable or via Bluetooth™, Wi-Fi, wireless, or Internet connection devices. The controller 12, thus, may communicate with the distributed sensor 11 via a wired or a wireless connection.

FIG. 3D illustrates an inner view of the exemplary pad 5 including the distributed sensor 11. The distributed sensor 11 is uniformly distributed over an inner surface of the pad 5, i.e., imbedded within the pad. In another embodiment, the distributed sensor 11 may instead be distributed over an outer surface of the pad 5.

FIG. 3E illustrates an inner view of a second exemplary pad 5′ that differs from the exemplary pad 5 of FIG. 3D in that the pad 5′ includes an accelerometer 26 located within the pad 5′. The accelerometer 26 may register distance of the chest movement caused by a chest compression. Thus, this embodiment allows sensing of chest compression parameter (e.g., depth of chest compression) by using both the distributed sensor 11 and an accelerometer 26. Typically, an accelerometer alone may not sense accurate chest displacement during chest compressions since the accelerometer does not detect any force lingering on the chest of the patient after a chest compression. That is, an accelerometer simply measures instantaneous displacement without accounting for a situation in which a total recoil has not yet occurred. Thus, a trainee using a CPR device having an accelerometer only for chest depth measurement may read 3-centimeter distance measured by the accelerometer, but would not be aware that the chest may still have a compressed distance of 2 cm due to a lingering chest compression force. Such a rescuer may inadvertently apply more force for compression, thereby resulting in ineffective CPR or an injury to the patient.

The present disclosure, however, provides a distributed sensor 11 that may still sense any lingering force remaining on the surface area after a chest compression. The distributed sensor 11 then transmits the sensed lingering force to the controller 12. The controller 12, in turn, calculates the correct amount of the chest compression depth taking account of the depth caused by the lingering force. The controller 12 then produces performance information including the accurate chest compression depth to be displayed on the display 17. The controller 12 may also produce performance information indicating that a total recoil has not yet occurred. The prompting device 13, in turn, may provide performance information by turning on a light emitting element 16, indicating that the total recoil has not yet occurred. Thus, the accelerometer 26 used together with the distributed sensor 11 and performance information based on data from the accelerometer 26 and the distributed sensor 11 may facilitate recognizing a predefined value of the depth of the chest compression by a trainee.

FIG. 3F illustrates a front view of a third exemplary pad 5″ which includes a light emitting element 16′ arranged on the surface area of the pad 5″. The light emitting element 16′ may perform similar functions as the light emitting element 16 of the prompting device 13. The pad 5″ may also have on it instructions 23 on how to administer CPR or any other information that may be helpful to a CPR trainee.

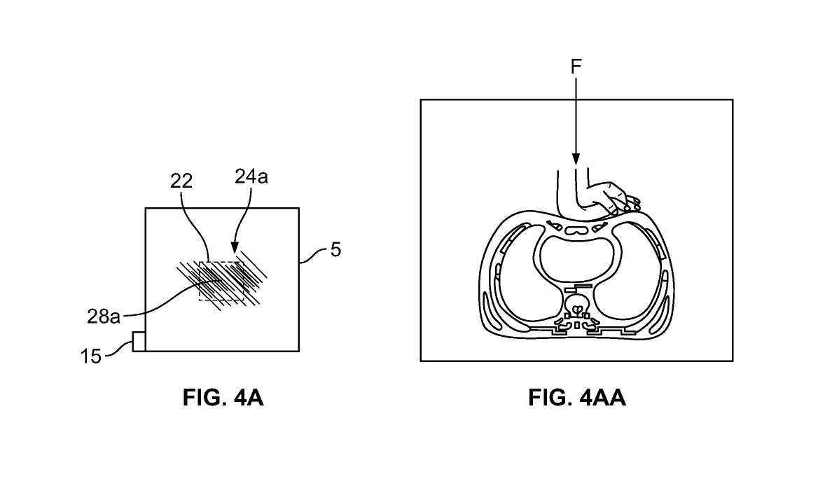

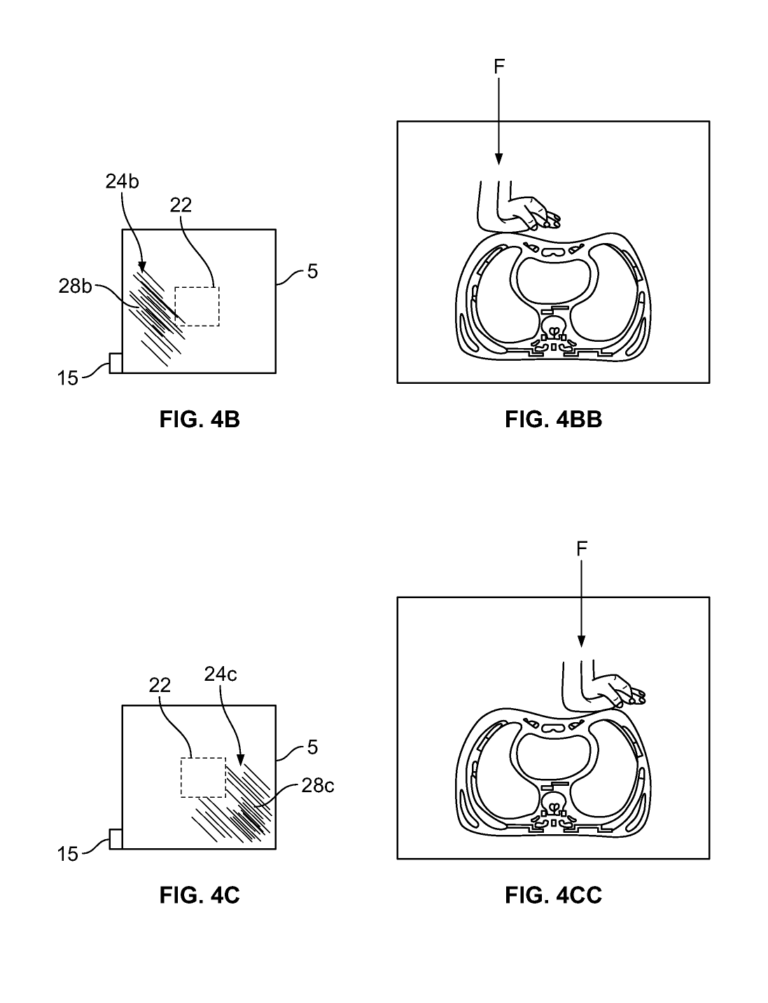

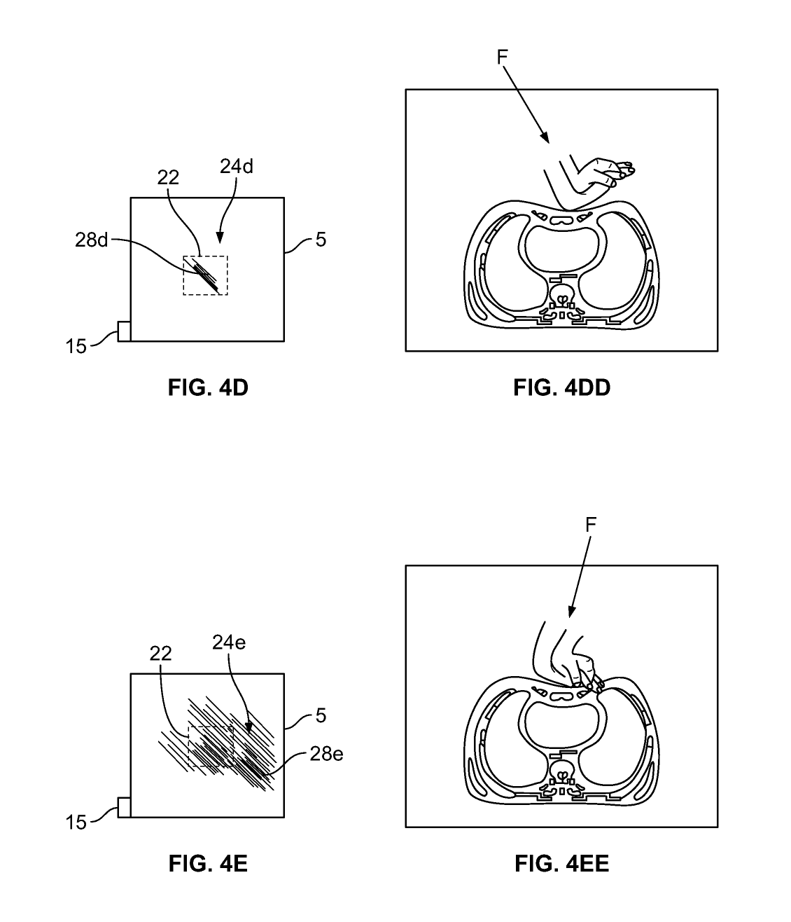

FIG. 4A illustrates a proper location of a force applied 24 for chest compression throughout a surface of area of a pad 5 having a distributed sensor 11. The detected location of force illustrated in FIG. 4A corresponds to the technique shown in FIG. 4AA in which the force F is being applied directly above the sternum of the patient and not an angle.

FIG. 4A shows the force applied 24a throughout the surface area as sensed by the distributed sensor 11. As shown in FIG. 4A, the force applied 24a is distributed throughout the surface area around the center of mass 28a of the trainee's exerted force 24a. A center of mass is a defined term and may mean an arithmetic mean of all points weighted by a local density or specific weight. In FIG. 4A, the center of mass 28a resides in the center 22 of the surface area of the pad 5 which is vertically aligned with the target CPR location, the sternum point at which the lower rib cages meet. Thus, the force 24a is being applied at the proper location. Aligning the center of mass 28a of the trainee's exerted force 24a and the center 22 of the pad 5 results in correct vertical alignment of the chest compression force 24.

In addition, the force applied 24a is in a shape generally resembling a palm of a person's hand. The force applied 24a is generally evenly distributed along the center 22, even if some of the force is outside the center 22. A force applied 24a that is evenly distributed along the center 22 corresponds to proper CPR hand form in which the trainee is using most of her palm to exert the force F and not just the heel of her hand. The pad 5 may detect such proper form by detecting that the force is evenly distributed along the center 22, even if some of the force is outside the center 22.

FIGS. 4B-4CC illustrate improper locations of the chest compression force 24 throughout the surface area of the pad 5 as sensed by the distributed sensor 11. In FIGS. 4B and 4C the pad 5 remains at the same location with respect to the patient as in FIG. 4A. The center 22 of the surface area of the pad 5 is vertically aligned with the target CPR location, the sternum point at which the lower ribs of the patient meet.

FIG. 4B shows the force 24b applied towards the left of the surface area, thereby misaligning the center of mass 28b and the center 22 of the pad 5. The detected location of force illustrated in FIG. 4B corresponds to the technique shown in FIG. 4BB in which the force F is being applied, not directly above the sternum of the patient, but to the side. FIG. 4C shows the force 24c applied towards the bottom right corner of the surface area, thereby misaligning the center of mass 28c and the center 22 of the pad 5. The detected location of force illustrated in FIG. 4C corresponds to the technique shown in FIG. 4CC in which the force F is being applied, not directly above the sternum of the patient, but to the side.

FIGS. 4D-4EE illustrate improper angles or hand form of the chest compression force 24 throughout the surface area of the pad 5 as sensed by the distributed sensor 11. In FIGS. 4D and 4E the pad 5 remains at the same location with respect to the patient as in FIG. 4A. The center 22 of the surface area of the pad 5 is vertically aligned with the target CPR location, the sternum point at which the lower ribs of the patient meet.

FIG. 4D shows the force 24d whose center of mass 28d resides in the center 22 of the surface area of the pad 5. In FIG. 4A this corresponded to proper form but in FIG. 4D notice that the force 24d is not distributed evenly along the center 22. The force 24d is instead very concentrated around the center of mass 28d. This is an indication that the trainee is not using proper form. The force applied 24d is not in a shape resembling a palm of a person's hand. The force applied 24d instead resembles a portion of the heel of the trainee's hand. A force applied 24d that is not evenly distributed along the center 22 corresponds to improper CPR hand form as shown in FIG. 4DD in which the trainee is not using most of her palm or even most of her heel to exert the force F but mostly a portion of the heel of her hand. As a result, the force F is too heavily concentrated on one spot.

The force F in FIG. 4DD is also being applied at an angle, which is also improper as angled compression of the chest may not compress the heart, which is essential for proper CPR. Notice the difference between the directly-over-the-patient force F of FIG. 4AA and the force F of FIG. 4DD which is applied at an angle. The pad 5 may detect such improper form or angle by detecting that the force 24d is not evenly distributed along the center 22 even if its center of mass 28d appears in the center 22. The force 24d is heavily concentrated along the center 22 instead, which the pad 5 may detect as improper. For example, the pad 5 may require that the force 24d be distributed over a minimum area (e.g., 2 in², 3 in², 4 in², etc.)

FIG. 4E shows the force 24e whose center of mass 28e resides outside the center 22 of the surface area of the pad 5 even though a significant portion of the force 24e resides within the center 22. This is an indication that the trainee is not using proper form. The force applied 24e is not in a shape resembling a palm of a person's hand but in a shape resembling the trainee's whole hand. A force applied 24e such as that of FIG. 4E indicates that the trainee may be too far over the patient and thus the exerted force is too heavily exerted by the trainee's fingers. As a result, the force F appears too heavily away from the proper location. The force F appears on the patient's chest as if it is being applied at an angle, which is also improper. Notice the difference between the directly-over-the-patient force F of FIG. 4AA and the force F of FIG. 4EE which is applied at an angle. The pad 5 may detect such improper form or angle by detecting that the force 24e is distributed along too large of an area or by detecting that the center of mass 28e appears outside the center 22. For example, in addition to requiring that the center of mass 28e appear in the center 22, the pad 5 may require that the force 24e be distributed over a maximum area (e.g., 18 in², 19 in², 20 in², etc.)

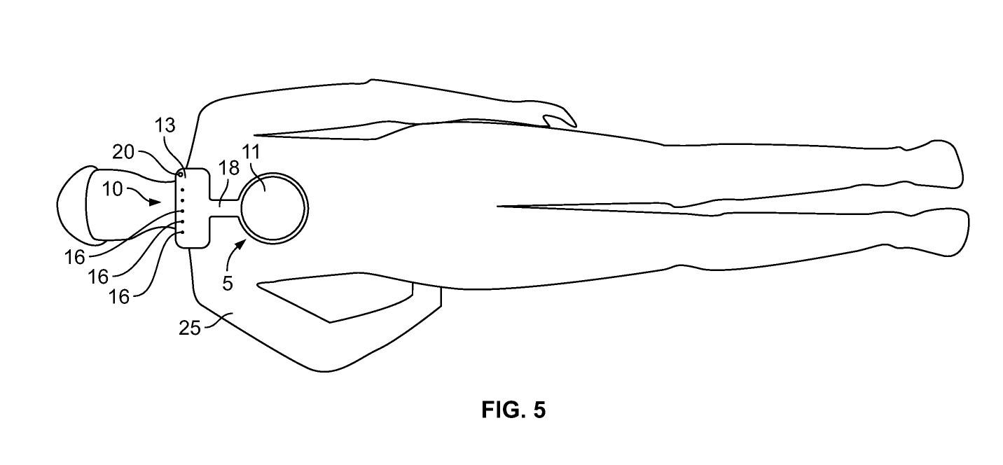

FIG. 5 illustrates an in-use view of the exemplary CPR training apparatus 10. The pad 5 with the distributed sensor 11 is placed above a chest area of a patient 25. As described above, the pad 5 includes the distributed sensor 11 and the placement aide 14 formed on the bottom side of the pad 5 and shaped similar to a positive image of a person's costal angle of the xyphoid process of the sternum or chest. Thus, the pad 5 may be consistently placed correctly over the patient's chest area so that the center of the surface area of the pad 5 is vertically aligned with the target sternum point SP. The light emitting elements 16 and audio device 20 may notify the trainee of the performance information during chest compressions. The distributed sensor 11 in pad 5 register if pressure is applied at an angle and not vertically or at an improper location. With this information, the trainee can make an appropriate corrective measure by simply moving his hands and body towards the correction form and location.

The CPR training apparatus 10 may also be used on a hard surface, instead of on a person 25. In addition, the apparatus 10 of the present disclosure dispenses with the need to use a mannequin the size of a person 25 as required by conventional CPR training devices. Such a life-sized mannequin is generally cumbersome to transport. However, the apparatus 10 due to its relatively small size is easy to transport, making CPR training using the apparatus 10 more convenient for the user. Because of the configuration of the distributed sensor 11 uniformly distributed over the pad 5, the distributed sensor 11 is capable of sensing the chest compression force throughout the surface area even when placed on a hard surface. As described above, the amount and location of force may provide information useful for CPR training and this information is available even when the device 10 is placed on a hard surface. For example, we may know that a 29.5 kg chest compression force corresponds to the preferred 5 cm chest compression depth. The controller 12 may compare the measured amount of chest compression force and the predefined amount of 29.5 kg. The prompting device 13 may then provide the trainee performance information regarding the sufficiency of the amount of the force applied. In another example, location and angle of force exerted may be detected as described above even when the device 10 is used on a hard surface. Therefore, the apparatus 10 may allow CPR training to be conducted in a large variety of locations making CPR training more easily available.

Returning to FIG. 3E, as described above, the distributed sensor 11 may be used in combination with the accelerometer 26 to provide more accurate feedback as to proper CPR technique. In some scenarios, however, the accelerometer 26 may not sense accurate chest displacement during chest compressions. For example, if the person 25 of FIG. 5 is lying on a bed or similar elastic or yielding surface, the accelerometer 26 will likely not sense accurate chest displacement during chest compressions. This is because a significant amount of force applied to the chest will result in displacement of the surface (e.g., bed's mattress) and not on displacement of the chest of the person 25. The accelerometer 26 may, for example, read 10 or 15 cm of displacement when, in reality, the chest of the person 25 is not experiencing this relatively high level of displacement.

In one embodiment, the CPR training apparatus 10 may be configured to, when receiving a displacement measurement from the accelerometer 26 that exceeds a predetermined threshold, disregard the displacement measurement received from the accelerometer 26. In one embodiment, the CPR training apparatus 10 is configured to, when receiving a displacement measurement from the accelerometer 26 that exceeds a predetermined threshold of 10 cm, disregard the displacement measurement received from the accelerometer 26. In one embodiment, the CPR training apparatus 10 is configured to, when receiving a displacement measurement from the accelerometer 26 that exceeds a predetermined threshold of 5 cm, disregard the displacement measurement received from the accelerometer 26. In yet other embodiments, the CPR training apparatus 10 is configured to, when receiving a displacement measurement from the accelerometer 26 that exceeds a predetermined threshold in the range from 5 cm to 15 cm, disregard the displacement measurement received from the accelerometer 26.

In such embodiment, once the CPR training apparatus 10 disregards the displacement measurement received from the accelerometer 26, the apparatus 10 may rely solely or mostly on a measurement received from the distributed sensor 11. Thus, for example, if the displacement measurement received from the accelerometer 26 exceeds the predetermined threshold because the person 5 is lying on a bed while receiving CPR, the controller 12 may disregard the displacement measurement received from the accelerometer 26 and rely on the measurement received from the distributed sensor 11.

In another embodiment, the controller 12 may first calculate a first calculated depth of the chest compression based at least in part on the sensed chest compression force from the sensor 11 and the measured depth of the chest compression from the accelerometer 26. The controller 12 may then compare the first calculated depth of the chest compression with a predetermined value for the depth of the chest compression and, when the first calculated depth of the chest compression exceeds the predetermined value for the depth of the chest compression, disregard the measured depth of the chest compression from the accelerometer 26. The controller 12 may then calculate a second calculated depth of the chest compression based at least in part on the sensed chest compression force from the sensor 11.

The CPR apparatus 10 may provide feedback that the compression force being applied is not sufficient if the force is measured to be less than a predetermined amount and provide feedback that the compression force being applied is too much if the force is measured to be more than the same or a different predetermined amount. The controller 12 may compare the measured amount of chest compression force and the predefined amount. The prompting device 13 may then provide performance information regarding the sufficiency of the amount of the force applied. For example, we may know that a 29.5 kg chest compression force corresponds to the preferred 5 cm chest compression depth. Thus, the CPR apparatus 10 may provide feedback that the compression force being applied is not sufficient if the force is measured to be less than 29.5 kg or provide feedback that the compression force being applied is too much if the force is measured to be more than 29.5 kg.

This way the CPR apparatus 10 may be used even on a soft, elastic, or yielding surface such as a bed. Therefore, the apparatus 10 may allow CPR training to be conducted in a large variety of locations making CPR training more easily available.

While example systems, methods, and so on, have been illustrated by describing examples, and while the examples have been described in considerable detail, it is not the intention to restrict or in any way limit the scope of the appended claims to such detail. It is, of course, not possible to describe every conceivable combination of components or methodologies for purposes of describing the systems, methods, and so on, described herein. Additional advantages and modifications will readily appear to those skilled in the art. Therefore, the invention is not limited to the specific details, and illustrative examples shown or described. Thus, this application is intended to embrace alterations, modifications, and variations that fall within the scope of the appended claims. Furthermore, the preceding description is not meant to limit the scope of the invention. Rather, the scope of the invention is to be determined by the appended claims and their equivalents.

To the extent that the term “includes” or “including” is employed in the detailed description or the claims, it is intended to be inclusive in a manner similar to the term “comprising” as that term is interpreted when employed as a transitional word in a claim. Furthermore, to the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (3D. Ed. 1995).

What is claimed is:

1. A cardiopulmonary resuscitation training apparatus comprising:
a distributed sensor configured to sense chest compression force;
an accelerometer configured to measure a depth of a chest compression; and
a controller operatively coupled to the distributed sensor and the accelerometer and configured to receive the sensed chest compression force from the distributed sensor and the measured depth of the chest compression from the accelerometer, the controller further configured to perform at least one of:
a) calculate a first calculated depth of the chest compression based at least in part on the sensed chest compression force and the measured depth of the chest compression, compare the first calculated depth of the chest compression with a predetermined value for the depth of the chest compression and, when the first calculated depth of the chest compression exceeds the predetermined value for the depth of the chest compression, disregard the measured depth of the chest compression from the accelerometer and calculate a second calculated depth of the chest compression based at least in part on the sensed chest compression force, or
b) compare the measured depth of the chest compression from the accelerometer to a predetermined threshold for the depth of the chest compression and, when the measured depth of the chest compression from the accelerometer exceeds the predetermined threshold for the depth of the chest compression, disregard the measured depth of the chest compression from the accelerometer and calculate the second calculated depth of the chest compression based at least in part on the sensed chest compression force,
the controller further configured to produce performance information which is based on the second calculated depth of the chest compression.

2. The apparatus of claim 1, wherein the predetermined value for the depth of the chest compression is in the range of 5-15 cm.

3. The apparatus of claim 1, comprising:
a wired or wireless transmitter configured to receive and transmit the performance information.

4. The apparatus of claim 1, wherein the first calculated depth of the chest compression is calculated by averaging results of a) a calculation or a look up table in which the sensed chest compression force from the distributed sensor is correlated to an expected depth of chest compression for the sensed chest compression force from the distributed sensor and b) the measured depth of the chest compression from the accelerometer.

5. The apparatus of claim 1, wherein the second calculated depth of the chest compression is calculated by a calculation or a look up table in which the sensed chest compression force from the distributed sensor is correlated to an expected depth of chest compression for the sensed chest compression force from the distributed sensor.

6. The apparatus of claim 1, comprising:
a prompting device operatively coupled to the controller and configured to provide the performance information, wherein the prompting device includes a light or audio emitting device configured to indicate that:
an amount of the sensed chest compression force is less than a predefined amount of chest compression force to be applied for a chest compression;
the amount of the sensed chest compression force is more than the predefined amount of the chest compression force to be applied for the chest compression;
the amount of the sensed chest compression force is equal to the predefined amount of the chest compression force to be applied for the chest compression; and
the amount of the sensed chest compression force is zero.

7. The apparatus of claim 1, comprising:
a prompting device operatively coupled to the controller and configured to provide the performance information, wherein the prompting device includes a light or audio emitting device configured to indicate that:
the first or second calculated depth of the chest compression is less than a predefined amount of chest compression depth to be applied for a chest compression;
the first or second calculated depth of the chest compression is more than the predefined amount of the chest compression depth to be applied for the chest compression;
the first or second calculated depth of the chest compression is equal to the predefined amount of the chest compression depth to be applied for the chest compression; and
the first or second calculated depth of the chest compression is zero.

8. The apparatus of claim 1, comprising:
a prompting device operatively coupled to the controller and configured to provide the performance information, wherein the prompting device includes a light or audio emitting device configured to indicate a point in time for administering rescue breathing during chest compressions.

9. The apparatus of claim 1, comprising:
a prompting device operatively coupled to the controller and configured to provide the performance information, wherein the prompting device includes an audio device configured to produce:
a first audible prompt when a frequency of chest compressions is equal to a predefined frequency of chest compressions;
a second audible prompt different from the first audible prompt when the frequency of the chest compressions is higher than the predefined frequency; and
a third audible prompt different from the first audible prompt and the second audible prompt when the frequency of the chest compressions is lower than the predefined frequency.

10. The apparatus of claim 1, wherein the distributed sensor is configured for use on a patient lying on an elastic surface.

11. A method for cardiopulmonary resuscitation training, the method comprising:
sensing a chest compression force of a chest compression via a distributed sensor;
measuring a depth of the chest compression via an accelerometer;
performing at least one of:
a) calculate a first calculated depth of the chest compression based at least in part on the sensed chest compression force and the measured depth of the chest compression, compare the first calculated depth of the chest compression with a predetermined value for the depth of the chest compression and, when the first calculated depth of the chest compression exceeds the predetermined value for the depth of the chest compression, disregard the measured depth of the chest compression from the accelerometer and calculate a second calculated depth of the chest compression based at least in part on the sensed chest compression force, or
b) compare the measured depth of the chest compression from the accelerometer to a predetermined threshold for the depth of the chest compression and, when the measured depth of the chest compression from the accelerometer exceeds the predetermined threshold for the depth of the chest compression, disregard the measured depth of the chest compression from the accelerometer and calculate the second calculated depth of the chest compression based at least in part on the sensed chest compression force,
producing performance information which is based on the second calculated depth of the chest compression; and
providing the performance information.

12. The method of claim 11, wherein the predetermined value for the depth of the chest compression is in the range of 5-15 cm.

13. The method of claim 11, comprising:
wirelessly transmitting the performance information.

14. The method of claim 11, wherein the first calculated depth of the chest compression is calculated by averaging results of a) a calculation or a look up table in which the sensed chest compression force from the distributed sensor is correlated to an expected depth of chest compression for the sensed chest compression force from the distributed sensor and b) the measured depth of the chest compression from the accelerometer.

15. The method of claim 11, wherein the second calculated depth of the chest compression is calculated by a calculation or a look up table in which the sensed chest compression force from the distributed sensor is correlated to an expected depth of chest compression for the sensed chest compression force from the distributed sensor.

16. The method of claim 11, wherein the providing includes a light or audio emitting device indicating:
an amount of the sensed chest compression force is less than a predefined amount of chest compression force to be applied for a chest compression;
the amount of the sensed chest compression force is more than the predefined amount of the chest compression force to be applied for the chest compression;
the amount of the sensed chest compression force is equal to the predefined amount of the chest compression force to be applied for the chest compression; and
the amount of the sensed chest compression force is zero.

17. The method of claim 11, wherein the providing includes a light or audio emitting device indicating:
the first or second calculated depth of the chest compression is less than a predefined amount of chest compression depth to be applied for a chest compression;
the first or second calculated depth of the chest compression is more than the predefined amount of the chest compression depth to be applied for the chest compression;
the first or second calculated depth of the chest compression is equal to the predefined amount of the chest compression depth to be applied for the chest compression; and
the first or second calculated depth of the chest compression is zero.

18. The method of claim 11, wherein the providing includes a light or audio emitting device indicating a point in time for administering rescue breathing during chest compressions.

19. The method of claim 11, wherein the providing includes an audio emitting device indicating:
a first audible prompt when a frequency of chest compressions is equal to a predefined frequency of chest compressions;
a second audible prompt different from the first audible prompt when the frequency of the chest compressions is higher than the predefined frequency; and
a third audible prompt different from the first audible prompt and the second audible prompt when the frequency of the chest compressions is lower than the predefined frequency.

20. The method of claim 11, comprising:
performing CPR on a patient lying on an elastic surface.
